(19) United States
(12) Patent Application Publication
(10) Pub. No.: US 2003/0032581 A1
(43) Pub. Date: Feb. 13, 2003

(54) Pharmaceuticals for treating obesity

(76) Inventors: Joel P. Berger, Hoboken, NJ (US); Thomas W. Doebber, Scotch Plains, NJ (US); Mark D. Leibowitz, San Diego, CA (US); David E. Moller, Bedminster, NJ (US); Ralph T. Mosley, Roselle, NJ (US); Richard L. Tolman, Menlo Park, CA (US); John Ventre, Nutley, NJ (US); Bei B. Zhang, Edison, NJ (US); Gaochao Zhou, Scotch Plains, NJ (US)

Correspondence Address: Merck & Co., Inc., Patent Department, P.O. Box 2000 - RY60-30, Rahway, NJ, 07065-0907, US

(21) Appl. No.: 10/241,106

(22) Filed: Sep. 11, 2002

Related U.S. Application Data
(62) Division of application No. 09/691,955, filed on Oct. 19, 2000.
(60) Provisional application No. 60/161,225, filed on Oct. 22, 1999.

(51) Int. Cl. A61K 31/00

(52) U.S. Cl. 514/1

(57) ABSTRACT

Compounds which are antagonists of strong PPAR-gamma agonists, such as rosiglitazone, and are also partial agonists of the PPAR-gamma receptor, are active agents for correcting or reducing obesity. For example, 1-(p-chlorobenzyl)-5-chloro-3-thiophenylindole-2-carboxylic acid, is characterized as being a potent and selective ligand for PPAR-gamma which has partial agonist (<30% maximal effects relative to rosiglitazone) and antagonist activity in cell-free and cell-based assays for the PPAR-gamma receptor. The compound is a potent agent for reducing obesity and insulin resistance in fat-fed C57BL/6J mice. This compound and other PPAR-gamma antagonists/partial agonists and pharmaceutically acceptable salts are effective in the treatment of obesity and/or diabetes and/or insulin resistance.

### FIELD OF THE INVENTION

This invention relates to obesity and methods of treating or preventing obesity. In addition, the invention relates to methods for treatment or prevention of insulin resistance, Type II diabetes, and lipid disorders.

### BACKGROUND OF THE INVENTION

Excessive weight, and in extreme cases obesity, is a widespread medical problem in the United States and elsewhere as the new millenium approaches. This may be due in part to sedentary life styles and poor diet (high in fats and carbohydrates), as well as to a genetic predisposition in many cases.

Pharmaceuticals have been marketed in the past to help control excessive weight and obesity. These have typically tried to achieve weight loss by reducing the appetite. Drugs used to reduce appetite have not been universally successful. Many are stimulants and have been abused, and others have had unexpected, and sometimes serious side effects (e.g., fen-phen). An approach that has so far not been exploited successfully is the development of pharmaceuticals that control excessive weight and obesity using a metabolic approach by modulation of receptors that can influence weight gain.

Peroxisome proliferator activated receptors (PPAR) have attracted considerable scientific attention in the last few years in part because of their usefulness in treating Type II (non-insulin dependent) diabetes (NIDDM). There are three different PPAR sub-types each of which responds to different ligands, each with different results: (1) PPAR-gamma is expressed at high levels in adipose tissue and regulates adipocyte differentiation. It has been a prime target in the search for insulin sensitizing agents that can be used in the treatment of NIDDM. Troglitazone, rosiglitazone, and pioglitazone are all antidiabetic agents that are known to be PPAR-gamma agonists. (2) PPAR-alpha regulates the metabolism of lipids. Fatty acids and the fibrate class of hypolipidemic drugs are PPAR-alpha agonists. PPAR-alpha agonists increase catabolic lipid metabolism, and therefore are beneficial in reducing serum lipids. Newer classes of antidiabetes drugs that are currently under development act simultaneously as PPAR-alpha and PPAR-gamma agonists. These are expected to benefit patients by improving insulin sensitivity through activation of PPAR gamma and by also improving the serum lipid profile by activating PPAR-alpha. Improvements in the serum lipid profile are expected to greatly reduce the likelihood that the diabetes patient will also develop atherosclerosis. (3) PPAR-delta is a third receptor sub-type, whose exact function is less well characterized.

Several patent applications and publications have suggested that PPAR-gamma antagonists or partial agonists may be effective in the treatment of obesity. See WO 96/40128, WO 97/10813, and J. Oberfield, et al., Proc. Nat. Acad. Sci. USA, Vol. 96, pp 6102-6106 (1999). None of these references or others to date have provided in vivo data showing that PPAR gamma ligands have an effect in treating obesity. Also, since PPAR-gamma agonists are used in the treatment of NIDDM, and obesity generally accompanies NIDDM, PPAR agonists are generally also claimed as useful in the treatment of obesity.

The broad statements that PPAR-gamma agonists, antagonists and/or partial agonists are useful in treating obesity are at best speculative, and may be supported only by the fact that PPAR-gamma agonists are known to promote in vitro fat cell differentiation and (under some circumstances) the accumulation of adipose tissue in vivo. Furthermore, optimal methods for identifying PPAR-gamma antagonists and/or partial agonists have not been defined nor have in vitro assay criteria been established that allow for the selection of compounds which have a high likelihood of in vivo anti-obesity efficacy. This is further complicated by the fact that it is not clear whether PPAR antagonists, if they are active in the treatment of obesity, would cause greater degrees of insulin resistance and exacerbation of diabetes or whether they would result in an improved metabolic profile.

### SUMMARY OF THE INVENTION

Compound I, 1-(p-chlorobenzyl)-5-chloro-3-thiophenylindole-2-carboxylic acid, having the structure below:

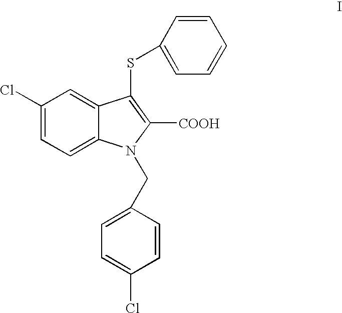

is characterized as being a potent and selective ligand for PPAR-gamma which has partial agonist (<30% maximal effects relative to a full agonist such as rosiglitazone) and antagonist activity in cell-free and cell-based assays described in this application for the PPAR-gamma receptor. Compound I is a potent agent for reducing obesity and insulin resistance in fat-fed C57B/6J mice. Compound I and other PPAR gamma antagonists/partial agonists (as defined in this invention), and pharmaceutically acceptable salts, are effective in the treatment of obesity and/or diabetes and/or insulin resistance in mice, other mammals, and humans in need of such treatment.

### BRIEF DESCRIPTION OF THE DRAWINGS

FIG. 1 is a graph that illustrates that Compound I antagonizes PPARγ agonist-induced PPARγ-CBP interaction in the HTRF assay.

FIG. 2 is a graph that illustrates that Compound I antagonizes PPARγ agonist-induced 3T3-L1 cell adipogenesis.

FIG. 3 is a graph of the body weight of mice fed a low fat diet, a high fat diet, and a high fat diet +50 mpk of Compound I.

FIG. 4 illustrates the epididymal fat pad weight of mice fed a low fat diet, a high fat diet, and a high fat diet+Compound 1 (50 mpk). Note that p<0.05 for the rats fed a 60% fat diet plus 50 mpk of compound I compared to the 60% fat control, and p<0.01 for the rats fed an 11% fat diet compared to the 60% fat control.

FIG. 5 illustrates the perirenal fat pad weight of mice fed a low fat diet, a high fat diet, and a high fat diet +Compound 1 (50 mpk).

FIG. 6 illustrates the % body lipid of mice fed a low fat diet, a high fat diet, and a high fat diet +Compound I. The % body lipid is determined from total carcass triglyceride as a % of carcass weight.

FIG. 7 illustrates the % of body protein in mice fed a low fat diet, a high fat diet, and a high fat diet+Compound I. Note that p<0.01 for the rats fed a 60% fat diet plus 50 mpk of Compound I compared to the 60% fat diet control.

### DETAILED DESCRIPTION OF THE INVENTION

Compounds that cause greater than 50% inhibition of the maximal agonism of a full PPAR-gamma agonist (i.e., a potent agonist, such as rosiglitazone, pioglitazone or 3-chloro-4-(3-(3-phenyl-7-propylbenzofuran-6-yloxy)propylthio)phenylacetic acid), and preferably greater than 75% inhibition, as measured by the PPAR-gamma-GAL4 transactivation assay or the homogeneous time-resolved fluorescence (HTRF) assay or other methods, described below, are potent compounds for treating obesity and insulin resistance. This is demonstrated in vivo by tests on fat-fed C57BL/6J mice. Compound I and many other PPAR-gamma antagonists exhibit partial agonism in addition to antagonism, and therefore the compounds that may be used for treating obesity and insulin resistance are described as PPAR-gamma antagonists/partial agonists, which includes both antagonists with partial agonism and no agonism.

Compounds that exhibit 100% antagonism (i.e., no agonism) can be used to treat obesity, but compounds like Compound I that have residual PPAR-gamma agonism and that are partial agonists in addition to being antagonists, may be particularly desirable because they are effective in treating not only obesity, but also in controlling hyperglycemia in individuals who need such control. The PPAR-gamma antagonists/partial agonists are therefore effective in treating the obesity and other symptoms that generally occur in non-insulin dependent diabetes, such as elevated levels of glucose, triglycerides, and insulin in the plasma. Experiments conducted with Compound I using ob/ob mice, a genetic model of extreme insulin resistance without overt hyperglycemia, have also demonstrated that markedly elevated blood insulin levels can be controlled. Thus, a substantial (30-50%) reduction in elevated levels of mean plasma insulin was observed following once daily dosing of ob/ob mice with Compound I in the absence of any effect on food intake. In agreement with the examples provided later in this application using C57BL/6J fat-fed mice, these results also establish the utility of the PPAR-gamma antagonists/partial agonists for improving insulin sensitivity in vivo in mammals.

The PPAR-gamma antagonists/partial agonists may also be effective in treating obesity that accompanies pre-diabetic conditions, where the patient does not have the blood sugar levels characteristic of type II diabetes (fasting glucose level of greater than 110-125 mg/dL), but still exhibits symptoms of insulin resistance and impaired glucose tolerance. This can result in better control of blood sugar as well as weight control, and may prevent or delay the onset of non-insulin dependent diabetes in an individual having a pre-diabetic condition.

As stated above, compounds that are effective in treating obesity and possibly other conditions inhibit the PPAR-gamma agonism of a full agonist, such as rosiglitazone, pioglitazone or 3-chloro-4-(3-(3-phenyl-7-propylbenzofuran-6-yloxy)propylthio)phenylacetic acid to a level of less than 50% of its normal level of agonism in a transactivation or HTRF assay, and preferably less than 25% of its normal level of agonism. Compounds that are effective in treating obesity and other conditions may also be characterized as exhibiting partial agonism in addition to antagonism, so that the antagonist exhibits agonism in the range of about 5% to about 50% of the normal level of agonism of the full agonist, and preferably agonism in the range of about 5% to about 25% of the agonism of the full agonist, in a transactivation or HTRF assay.

The above measurements of PPAR-gamma agonism and antagonism and their relative levels can be determined using the well known GAL4 chimeric receptor transcriptional assay, as described by Berger et al, Journal of Biological Chemistry, Vol 274, 6718-6725 (1999). A second means of measuring PPAR-gamma agonism is to use the PPAR-CBP HTRF assay, as described by Zhou, et al, Molecular Endocrinology, Vol. 12, 1594-1604 (1998), which reference is incorporated herein by reference, and in commonly assigned copending U.S. application Ser. No. 09/166,265, filed Oct. 5, 1998, now published as WO 99/18124, which is incorporated by reference into this application. Finally, the levels of agonism and antagonism can be measured using the 3T3-L1 pre-adipocyte differentiation assay, as described by Berger et al, Journal of Biological Chemistry, Vol 274, 6718-6725 (1999), with the compound being tested alone and in the presence of a full agonist in the assay. All of these are also described in the examples.

The following chemical compound and classes of compounds are useful in the treatment and prevention of obesity, and insulin resistance or NIDDM, or lipid disorders, and certain other conditions in mammals and human beings in need of such treatment.

Compound I, 1-(p-chlorobenzyl)-5-chloro-3-thiophenylindole-2-carboxylic acid, the structure of which is shown in the Summary of the Invention, is a PPAR-gamma antagonist/partial agonist. The data in the examples illustrate that when Compound I is included in the diets of mice that are consuming high fat levels, significant reductions in the accretion weight and body fat are achieved. The levels of plasma glucose, lipids and insulin are also improved (i.e., plasma glucose, triglycerides, free fatty acids and insulin are all reduced to more normal levels). Compound I is broadly included in a class of compounds having structures represented by Formula II below. Many of the compounds that are included in the scope of Formula II below, including pharmaceutically acceptable salts, will be PPAR-gamma antagonists/partial agonists. These compounds, including pharmaceutically acceptable salts, may also be active in treating obesity.

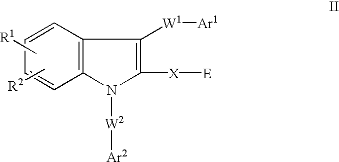

In Formula II, R¹ and R² are independently selected from H, halogen, C₁₋₁₀ alkyl, C₂₋₁₀ alkenyl and C₁₋₁₀ alkoxy, where the alkyl, alkenyl, and alkoxy groups are optionally substituted with 1-3 groups independently selected from R^{a}, except that the number of optional F groups when R^{a} is F is in the range of 1-21;

R^{a} is selected from OH, halogen, C₁₋₃ alkoxy, C₁₋₃ alkoxy having 1-7 halogen atom substituents, phenyl, and phenyl substituted with 1-3 groups independently selected from halogen, OCH₃, OCF₃, CH₃, and CF₃; (R^{a} may optionally also be selected from C₁₋₃ alkyl and C₁₋₃ alkyl having 1-7 halogen atom substituents in addition to the preceding choices listed herein);

Ar¹ and Ar² are each independently selected from the group consisting of aryl and heteroaryl, wherein Ar¹ and Ar² are optionally substituted with 1-3 substituents independently selected from R^{a} and are optionally substituted with one COOH group.

X, W¹ and W² are each independently selected from the group consisting of a single bond, Y, or Y(CH₂)ₙY¹, where Y and Y¹ are each independently selected from the group consisting of a single bond, O, S, SO, SO₂, and NR;

n is 1-3;

R is selected from H, C₁₋₃ alkyl and C₂₋₃ alkenyl, where the alkyl and alkenyl groups are optionally substituted with 1-7 halogen atoms and/or 1-3 groups selected from OH, C₁₃ alkoxy, and C₁₋₃ alkoxy substituted with 1-7 halogen atoms;

and E is CO₂H, C(O)NR₂, or a tetrazol-5-yl, where each R is independently defined above.

In a subset of compounds of Formula I,

R¹ is H, C₁₋₃ alkyl, C₁₋₃ alkoxy, or halogen;

R² is H;

W¹ and W² are each a single bond, O, S, SO, SO₂, NH, or CH₂;

X is a bond or CH_{2;}

Ar¹ and Ar² are each aryl, optionally substituted with 1-2 substituents independently selected from halogen, methoxy, and C₁₋₃ alkyl; and

E is CO₂H.

In another subset of compounds, heteroaryl is defined as thiophene, pyrrole, furan, or pyridine.

In preferred embodiments, aryl is phenyl.

A generic class of compounds is also presented as Formula I and Ia in U.S. Pat. Nos. 5,081,138, and 5,225,421, columns 2-5, and these generic formulas also may include compounds that are active in the treatment of obesity. U.S. Pat. Nos. 5,081,138 and 5,225,421 are incorporated by reference with this application in their entirety. Except for the claims, these patents appear identical. Definitions of substituents in Formula I and Ia of U.S. Pat. Nos. 5,081,138 and 5,225,421 are defined in those patents. Substituents in Formula II above and other words needing definition in this application are defined below.

The compounds that are described herein, including the generic group of compounds that include PPAR-gamma antagonists/partial agonists, are useful in treating, controlling, and preventing obesity, as well as many other diseases. These include but are not limited to:

(1) a method for treating, controlling, or preventing obesity in a mammal which comprises administering to the mammal a therapeutically effective amount of a compound described herein;

(2) a method for treating or controlling non-insulin dependent diabetes mellitus in a mammal which comprises administering to the mammal a therapeutically effective amount of a compound described herein;

(3) a method for treating, controlling or preventing hyperglycemia in a mammal which comprises administering to the mammal a therapeutically effective amount of a compound described herein;

(4) a method for treating, controlling or preventing hyperlipidemia in a mammal which comprises administering to the mammal a therapeutically effective amount of a compound described herein;

(5) a method for treating, controlling or preventing hypercholesterolemia in a mammal which comprises administering to the mammal a therapeutically effective amount of a compound described herein;

(6) a method for treating, controlling or preventing hypertriglyceridemia in a mammal which comprises administering to the mammal a therapeutically effective amount of a compound of described herein;

(7) a method for treating, controlling or preventing dyslipidemia in a mammal which comprises administering to the mammal a therapeutically effective amount of a compound described herein;

(8) a method for treating, controlling or preventing hyperinsulinemia in a mammal which comprises administering to the mammal a therapeutically effective amount of a compound described herein;

(9) a method for treating or controlling cancer in a mammal which comprises administering to the mammal a therapeutically effective amount of a compound described herein;

(10) a method for treating, controlling or preventing inflammatory bowel disease or inflammatory conditions in a mammal which comprises administering to the mammal a therapeutically effective amount of a compound described herein; and

(11) a method for treating, controlling or preventing insulin resistance in a mammal which comprises administering to the mammal a therapeutically effective amount of a compound described herein.

New Test Methods

A number of test methods that are described herein are new:

First, C57BL/16J mice have been shown to serve as an effective animal model to identify and characterize compounds that serve as antiobesity and antidiabetic agents. PPAR-gamma ligands are tested as anti-obesity and/or anti-diabetic agents in vivo by the steps of (1) administering a PPAR-gamma ligand to one or more C57BL/6J mice for a period of at least 14 days, and (2) measuring the effect of the PPAR-gamma ligand on one or more parameters that characterize obesity and/or diabetes. Preferably, the PPAR-gamma ligands are administered to the mice by feeding the ligands to the mice or administering by oral gavage. Typically, the experiments are carried out for more than two weeks, perhaps 1-3 months. Parameters that characterize obesity and/or diabetes that may be measured in the in vivo experiments include body weight, epididymal fat pad weight, perirenal fat pad weight, whole body triglyceride content, whole body protein content, body adiposity, lean body mass, plasma glucose level, plasma triglyceride level, plasma free fatty acid level, and serum insulin level.

The invention also comprises a method for selecting compounds for further testing (i.e. in vivo tests), for rapidly identifying new lead compounds from several candidates, and for screening large numbers of samples.

A method for selecting a compound for in vivo testing as an anti-obesity agent, comprises the steps of (1) providing a candidate compound, and (2) measuring the PPAR-gamma antagonism of the candidate compound in the presence of a full PPAR-gamma agonist. A full PPAR agonist is one that binds very effectively to the PPAR-gamma receptor and markedly induces transcriptional activation (in the Gal4-transactivation assay) or markedly promotes coactivator association (in the HTRF assay). Examples of full PPAR-gamma agonists include rosiglitazone and 3-chloro-4-(3-(3-phenyl-7-propylbenzofuran-6-yloxy)propylthio)phenylacetic acid.

The methods of screening for PPAR-gamma antagonists are suitable for large collections of samples, such as are found in combinatorial libraries. Samples being tested would then be part of a collection of at least 10 candidate compounds, and more typically could be part of a library of hundreds or thousands of compounds. The PPAR-CBP-HTRF assay is particularly suited to large-scale screening of large numbers of samples. For selecting a candidate for further testing for use as an anti-obesity compound, the compound or compounds which exhibit the highest % inhibition of the full PPAR-gamma agonist are generally selected for further testing as an anti-obesity agent. For example, at least one of the three samples having the highest inhibition of the full PPAR-gamma agonist would typically be tested further. Methods that are used to test for PPAR antagonism include assay methods selected from the group consisting of the PPAR-CBP HTRF assay, the GAL-4 chimeric receptor transcriptional assay, and 3T3-L1 preadipocyte differentiation.

Selection of compounds for in vivo or other testing as anti-obesity agents which also have anti-diabetic activity would be similar to the tests described above. These tests include the steps of (1) providing a candidate compound, and (2) measuring the PPAR-gamma antagonism/partial agonism of the candidate compound compared with a full PPAR-gamma agonist. Typical full PPAR-gamma agonists include 3-chloro-4-(3-(3-phenyl-7-propylbenzofuran-6-yloxy)propylthio)phenylacetic acid, rosiglitazone, and pioglitazone. The candidate compounds could be part of large libraries, such as combinatorial libraries, and would be included in collections of at least 10 candidate compounds. In this case, since compounds having activity for both the treatment of obesity and diabetes are being sought, the compounds that are likely to be selected for in vivo or other testing would be PPAR-gamma antagonists which also have a PPAR-gamma partial agonism in the range of about 5% to about 25% of the agonism of rosiglitazone or some other full agonist.

As was the case in evaluating antagonists, the methods used to measure PPAR-gamma partial agonism use an assay method selected from the group consisting of the PPAR-CBP HTRF assay, the GAL-4 chimeric receptor transcriptional assay, and 3T3-L1 preadipocyte differentiation. The PPAR-CBP-HTRF assay is preferred.

Finally, a method of measuring the partial agonism of a PPAR-gamma antagonist is provided. The method comprises the step of measuring the inhibition of a full agonist by a PPAR-gamma antagonist using the PPAR-CBP HTRF assay, and then measuring the residual agonism of the PPAR-gamma ligand during the same assay. The residual agonism of the PPAR-gamma antagonist is the partial agonism of the PPAR-gamma antagonist/partial agonist. Definitions “Alkyl”, as well as other groups having the prefix “alk”, such as alkoxy, alkanoyl, means carbon chains which may be linear or branched or combinations thereof. Examples of alkyl groups include methyl, ethyl, propyl, isopropyl, butyl, sec- and tert-butyl, pentyl, hexyl, heptyl, octyl, nonyl, and the like. A cycloalkyl group may be included in the alkyl group also, provided that the point of attachment is through the alkyl part of the group.

“Alkenyl” means carbon chains which contain at least one carbon-carbon double bond, and which may be linear or branched, or combinations thereof. Examples of alkenyl include vinyl, allyl, isopropenyl, pentenyl, hexenyl, heptenyl, 1-propenyl, 2-butenyl, 2-methyl-2-butenyl, and the like.

“Alkynyl” means carbon chains which contain at least one carbon-carbon triple bond, and which may be linear or branched or combinations thereof. Examples of alkynyl include ethynyl, propargyl, 3-methyl-1-pentynyl, 2-heptynyl and the like.

“Cycloalkyl” means mono- or bicyclic saturated carbocyclic rings, each having from 3 to 10 carbon atoms. The term also includes a monocyclic ring fused to an aryl group in which the point of attachment is on the non-aromatic portion. Examples of cycloalkyl include cyclopropyl, cyclopentyl, cyclohexyl, cycloheptyl, and the like.

“Aryl” (and “arylene”) means mono- or bicyclic aromatic rings containing only carbon ring atoms. The term also includes an aryl group fused to a monocyclic cycloalkyl or monocyclic heterocyclic group in which the point(s) of attachment is on the aromatic portion. The preferred aryl is phenyl.

“Heterocycle” and “heterocyclic” means a fully or partially saturated ring containing at least one heteroatom selected from N, S and 0, each of said rings having from 3 to 10 atoms. Examples of aryl include phenyl, naphthyl, indanyl, indenyl, tetrahydronaphthyl, benzopyranyl, 1,4-benzodioxanyl, and the like. Examples of heterocycles include tetrahydrofuran, piperazine, and morpholine.

“Aralkyl” means those radicals in which an aryl group is attached to an alkyl group, and the point of attachment is through the alkyl chain. The aryl group may also have alkyl substituents.

The terms “Obesity” and “Obese” generally refer to individuals whose body weight is at least 20% above the average body weight for the individual's age, gender and height. An individual is also defined as “obese” if the individual is a male whose body mass index is greater than 27.8 kg/m² or a female whose body mass index is greater than 27.3 kg/m². Those of skill in the art will recognize that individuals can be significantly above the average weight for their age, gender, and height and still technically not be “obese.” Such individuals are referred to as “overweight” herein, in accordance with normal usage. This invention will be beneficial for such overweight individuals, and may also be beneficial to individuals who are prone to obesity or to being overweight and who wish to avoid a recurrence of earlier episodes of obesity or being overweight.

“Heteroaryl” (and heteroarylene) means a mono- or bicyclic aromatic ring containing at least one ring heteroatom selected from N, O and S (including SO and SO₂), with each ring containing 5 to 6 atoms. Examples of heteroaryl include pyrrolyl, isoxazolyl, isothiazolyl, pyrazolyl, pyridyl, oxazolyl, oxadiazolyl, thiadiazolyl, thiazolyl, imidazolyl, triazolyl, tetrazolyl, furanyl, triazinyl, thienyl, pyrimidyl, pyridazinyl, pyrazinyl, benzisoxazolyl, benzoxazolyl, benzothiazolyl, benzimidazolyl, benzofuranyl, benzothiophenyl (including S-oxide and dioxide), furo(2,3-b)pyridyl, quinolyl, indolyl, isoquinolyl, dibenzofuran and the like. Preferred heteroaryls include pyrrole, thiophene, pyridine and furan.

“Halogen” includes fluorine, chlorine, bromine and iodine.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

The terms “PPAR-gamma antagonist” and “PPAR-gamma antagonist/partial agonist” both mean a compound that reduces the activity of a very effective (“full”) agonist of the PPAR-gamma receptor, such as rosiglitazone, pioglitazone or 3-chloro-4-(3-(3-phenyl-7-propylbenzofuran-6-yloxy)propylthio)phenylacetic acid (described by Berger et al, Journal of Biological Chemistry Vol 274, 6718-6725, 1999) to less than 50% of its normal activity, and preferably to less than 25% of its normal activity, as measured by the HTRF assay, by 3T3-L1 preadipocyte differentiation, or by the PPAR-gamma-GAL4 chimeric receptor transactivation assay. The HTRF assay is preferred.

“PPAR-gamma antagonism” means inhibition of the activity of a very effective (full) agonist of the PPAR-gamma receptor, usually measured as a % of inhibition of the agonism of the full agonist, generally to less than half of the activity of the full agonist, often to 25% of the activity of the full agonist. A compound that has “PPAR-gamma partial agonism” reduces the activity of a full agonist to the range of less than 50% of its activity down to about 5% of its normal activity (i.e. it is an antagonist); in this range, the residual activity, stated as a % of the activity of the full agonist, is attributed to partial agonism of the antagonist/partial agonist. The partial agonism and the antagonism by this definition usually add up to 100%. The antagonism and partial agonism are measured by the HTRF assay, by 3T3-L1 preadipocyte differentiation, or by the PPAR-gamma-GAL4 chimeric receptor transactivation assay, with the HTRF assay binding preferred.

Synthetic Methods

A synthesis of Compound I is described in U.S. Pat. No. 5,081,138. Other compounds generically described in U.S. Pat. No. 5,081,138 can also be made by the methods described in that patent. Compounds having Formula II can also be made by methods taught in U.S. Pat. No. 5,081,138 and by other methods well known to those of skill in the art of organic synthesis.

The synthesis of 3-chloro-4-(3-(3-phenyl-7-propylbenzofuran-6-yloxy)propylthio) phenylacetic acid is described in U.S. Pat. No. 5,859,051.

Optical Isomers—Diastereomers—Geometric Isomers—Tautomers—Prodrugs

Compounds of Formula II may contain one or more asymmetric centers and can thus occur as racemates and racemic mixtures, single enantiomers, diastereomeric mixtures and individual diastereomers. The present invention is meant to comprehend all such isomeric forms of the compounds of Formula II.

Some of the compounds described herein contain olefinic double bonds, and unless specified otherwise, are meant to include both E and Z geometric isomers.

Some of the compounds described herein may exist with different points of attachment of hydrogen, referred to as tautomers. Such an example may be a ketone and its enol form, known as keto-enol tautomers. The individual tautomers as well as mixtures thereof are encompassed with compounds of Formula II.

Compounds of Formula II that are diastereomers may be separated into diastereoisomeric pairs of enantiomers by, for example, fractional crystallization from a suitable solvent, for example methanol or ethyl acetate or a mixture thereof. The pair of enantiomers thus obtained may be separated into individual stereoisomers by conventional means, for example by the use of an optically active acid as a resolving agent.

Alternatively, any enantiomer of a compound of the general Formula II may be obtained by stereospecific synthesis using optically pure starting materials or reagents of known configuration.

The compound of formula I and the compounds of formula II, including carboxylate or other salts in solution, may be formed in the body from precursor compounds, called prodrugs, during or after administration, by some kind of conversion, such as a chemical reaction or metabolism. The prodrugs that yield the compounds of Formula I and II, including salts in solution, are also claimed as part of this invention. Non-limiting examples of prodrugs of the carboxylic acids of this invention would be esters of the carboxylic acid group, for example C₁ to C₆ esters, which may be linear or branched, and esters which have functionality that makes them more easily hydrolyzed after administration to a patient.

Salts

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, and tartaric acids.

It will be understood that, as used herein, references to the compounds of Formula I and II are meant to also include the pharmaceutically acceptable salts.

Utilities

Compounds described herein are potent PPAR-gamma antagonists/partial agonists. As such, the compounds described herein, and PPAR-gamma antagonists/partial agonists in general, are particularly useful in treating, preventing, and controlling obesity, and are also useful for eliminating excess weight in overweight individuals. These uses are accomplished by the administration of a therapeutically effective amount of Compound I, of compounds defined by Formula II herein, of compounds that fall within the scope of Formula I and Ia of U.S. Pat. No. 5,081,138, columns 2-5, and other PPAR-gamma antagonists/partial agonists.

Because the compounds are partial agonists of PPAR-gamma, the compounds are also beneficial for treating or controlling numerous other conditions or diseases of mammals or of humans in need of such treatment. These conditions, disorders, diseases and the like in which the compounds described herein, and for which PPAR-gamma antagonists/partial agonists may be beneficial, include, in addition to obesity: (1) diabetes mellitus, (2) hyperglycemia, (3) hyperlipidemia, (4) hypertriglyceridemia, (5) hypercholesterolemia (including raising HDL levels), (6) atherosclerosis, (7) vascular restenosis, (8) irritable bowel syndrome or inflammatory bowel disease, (9) pancreatitis, (10) abdominal obesity, (11) adipose cell tumors, (12) adipose cell carcinomas such as liposarcoma, (13) inflammation, (14) dyslipidemia, (15) prostate cancer and other cancers, and (16) other disorders where insulin resistance is a component, including Syndrome X and ovarian hyperandrogenism (polycystic ovarian syndrome). The PPAR antagonists/partial agonists and compounds described herein may be used to treat these diseases or conditions separately, or may be used to treat them concurrently with the treatment of obesity.

Administration and Dose Ranges

Any suitable route of administration may be employed for providing a mammal, and especially a human, with an effective dosage of a compound of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. Preferably compounds of Formula I are administered orally.

The effective dosage of active ingredient employed may vary depending on the particular compound employed, the mode of administration, the condition being treated and the severity of the condition being treated. Such dosage may be ascertained readily by a person skilled in the art.

When treating, preventing or controlling obesity, overweight conditions, and other diseases for which compounds described herein are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

Pharmaceutical Compositions

Another aspect of the present invention provides pharmaceutical compositions which comprise: (1) the compound of Formula I, compounds of Formula II, compounds from U.S. Pat. No. 5,081,138, or other PPAR-gamma antagonists/partial agonists, and (2) a pharmaceutically acceptable carrier. The pharmaceutical compositions of the present invention comprise compounds described herein as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

The compositions include compositions suitable for oral, rectal, topical, parenteral (including subcutaneous, intramuscular, and intravenous), ocular (ophthalmic), pulmonary (nasal or buccal inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

In practical use, the compounds described herein can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, hard and soft capsules and tablets, with the solid oral preparations being preferred over the liquid preparations.

Because of their ease of administration, tablets and capsules represent the most advantageous oral dosage unit form, in which case solid pharmaceutical carriers are obviously employed. If desired, tablets may be coated by standard aqueous or nonaqueous techniques. Such compositions and preparations should contain at least 0.1 percent of active compound. The percentage of active compound in these compositions may, of course, be varied and may conveniently be between about 2 percent to about 60 percent of the weight of the unit. The amount of active compound in such therapeutically useful compositions is such that an effective dosage will be obtained. The active compounds can also be administered intranasally as, for example, liquid drops or spray.

The tablets, pills, capsules, and the like may also contain a binder such as gum tragacanth, acacia, corn starch or gelatin; excipients such as dicalcium phosphate; a disintegrating agent such as corn starch, potato starch, alginic acid; a lubricant such as magnesium stearate; and a sweetening agent such as sucrose, lactose or saccharin. When a dosage unit form is a capsule, it may contain, in addition to materials of the above type, a liquid carrier such as a fatty oil.

Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor.

Compounds described herein may also be. administered parenterally. Solutions or suspensions of these active compounds can be prepared in water suitably mixed with a surfactant such as hydroxy-propylcellulose. Dispersions can also be prepared in glycerol, liquid polyethylene glycols and mixtures thereof in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), suitable mixtures thereof, and vegetable oils.

Combination Therapy

Compounds described herein may be used in combination with other drugs that may also be useful in the treatment, prevention, suppression or amelioration of the diseases or conditions for which the compounds described herein are useful. Such other drugs may be administered, by a route and in an amount commonly used therefor, contemporaneously or sequentially with a compound described herein. When a compound of this invention is used contemporaneously with one or more other drugs, a pharmaceutical composition in unit dosage form containing such other drugs and the compound of this invention is preferred. It is also contemplated that when used in combination with one or more other active ingredients, the compound of the present invention and the other active ingredients may be used in lower doses than when each is used singly. Accordingly, the pharmaceutical compositions of the present invention include those that contain one or more other active ingredients, in addition to a compound of this invention.

Examples of other active ingredients that may be combined with a compound of this invention, either administered separately or in the same pharmaceutical compositions, include, but are not limited to:

(a) insulin sensitizers including (i) PPARγ agonists such as the glitazones (e.g. troglitazone, pioglitazone, englitazone, MCC-555, rosiglitazone, and the like), and compounds disclosed in WO97/27857, 97/28115, 97/28137 and 97/27847; (ii) biguanides such as metformin and phenformin;

(b) insulin or insulin mimetics;

(c) sulfonylureas such as tolbutamide and glipizide, or related materials;

(d) α-glucosidase inhibitors (such as acarbose),

(e) cholesterol lowering agents such as (i) HMG-CoA reductase inhibitors (lovastatin, simvastatin and pravastatin, fluvastatin, atorvastatin, cerivastatin and other statins), (ii) sequestrants (cholestyramine, colestipol and a dialkylaminoalkyl derivatives of a cross-linked dextran), (iii) nicotinyl alcohol, nicotinic acid or a salt thereof, (iv) PPARα agonists such as fenofibric acid derivatives (gemfibrozil, clofibrate, fenofibrate and bezafibrate), (v) inhibitors of cholesterol absorption, for example beta-sitosterol and acyl CoA:cholesterol acyltransferase inhibitors, for example melinamide and (vi) probucol;

(f) PPARδ agonists such as those disclosed in WO97/28149;

(g) antiobesity compounds such as sulbitramine, orlistat, neuropeptide Y5 inhibitors, and β₃ adrenergic receptor agonists; and

(h) ileal bile acid transporter inhibitor.

### EXAMPLES

Methods

PPAR-Gamma Binding Assay

GST-hPPARγ fusion proteins were generated in E.coli (BL21 strain, Stratagene, La Jolla, Calif.). Cells were cultured in LB medium (GIBCO BRL, Gaithersburg, Md.) to a density of OD₆₀₀=0.7-1.0 and induced for overexpression by addition of isopropylthio-β-galactoside (IPTG) to a final concentration of 0.2 mM. The IPTG induced cultures were grown at room temperature for an additional 2-5 h, before cells were harvested by centrifugation for 10 min at 5000g. The GST-PPAR fusion protein was purified from the cell pellet using Glutathione Sepharose beads, following the procedure recommended by the manufacturer (Phamacia Biotech, Piscataway, N.J.).

For each assay, an aliquot of receptor, GST-hPPARγ diluted 1:1000-1:3000, was incubated in TEGM (10 mM Tris, pH 7.2, 1 mM EDTA, 10% glycerol, 7 μl/100 ml β-mercaptoethanol, 10 mM Na molybdate, 1 mM dithiothreitol, 5 μg/ml aprotinin, 2 μg/ml leupeptin, 2 μg/ml benzamide and 0.5 mM PMSF) containing 5-10% COS-1 cell cytoplasmic lysate and 10 nM of a [³H]₂-labelled thiazolidinedione (21Ci/mmole), as described by Berger et al, Journal of Biological Chemistry, Vol 274, 6718-6725 (1999), ±test compound. Assays were incubated for ˜16 h at 4° C. in a final volume of 300 μl. Unbound ligand was removed by incubation with 200 μl dextran/gelatin-coated charcoal, on ice, for μ10 minutes. After centrifugation at 3000 rpm for 10 min at 4° C., 200 μl of the supernatant fraction was counted in a liquid scintillation counter.

PPAR-Gamma-GAL4 Transactivation Assay

This assay was performed as described in Berger et al, Journal of Biological Chemistry, Vol 274, 6718-6725 (1999). In brief, COS-1 cells were transfected using Lipofectamine (GIBCO BRL, Gaithersburg, Md.) according to the instructions of the manufacturer. Transfection mixes for contained Lipofectamine, and a PPAR gamma-GAL4 chimeric expression vector, pcDNA3-PPARg/GAL4, pUAS(5X)-tk-luc reporter vector and 0.0002 μg of pCMV-lacZ as an internal control for transactivation efficiency. Cells were incubated in the transfection mixture for 5 h at 37° C. in an atmosphere of 10% CO₂. The cells were then incubated for ˜48 h in fresh high glucose DMEM containing 5% charcoal stripped fetal calf serum, nonessential amino acids, 100 units/ml Penicillin G and 100 mg/ml Streptomycin sulfate ± increasing concentrations of test compounds. Cell lysates were produced using Reporter Lysis Buffer (Promega, Madison, Wis.) according to the manufacturer's instructions. Luciferase activity in cell extracts was determined using Luciferase Assay Buffer (Promega, Madison, Wis.) in an ML3000 luminometer (Dynatech Laboratories, Chantilly, Va.). β-galactosidase activity was determined using β-D-galactopyranoside (Calbiochem, San Diego, Calif.).

PPAR-CBP HTRF Assay

HTRF assays were performed as previously described by Zhou et. al. (Molecular Endocrinology 12:1594-1604, 1998). Briefly, 100 mM HEPES, 123 mM KF, 0.125% (wt/vol) CHAPS, 0.05% dry milk, 1 nM GST-PPARγLBD, 2 nM anti-GST-(Eu)K, 10 nM biotin-CBP₁₋₄₅₃, 20 nM SA/XL665, a potent PPARγ agonist, Compound I (100 nM) and various concentrations of Compound I were incubated overnight at 4° C. Fluorescence was then read on a Discovery instrument (Packard). Data were expressed as the ratio, multiplied by a factor of 10⁴, of the emission intensity at 665 nM to that at 620 nM.

Measurement of 3T3-L1 Preadipocyte Differentiation

3T3-L1 cells were obtained from American Type Culture Collection. Passage numbers 3 to 9 were used in all the studies. Monolayer fibroblasts were maintained in medium A (Dulbecco's modified Eagle's medium with 10% fetal calf serum, 100 units/ml penicillin, and 100 μg/ml streptomycin) at 37° C. in 5% CO₂. For experiments, the cells were incubated with medium A (supplemented with 150 nM insulin, 1 μM dexamethasone) in the presence of 100 nM rosiglitazone and various concentrations of Compound I for 5 days (with one medium change). Total RNA was prepared using Ultraspec™ RNA isolation system (Biotecx, Houston, Tex.). RNA concentration was quantitated by absorbance at 260 nm. Equal amount of RNA samples were denatured in formamide/formaldehyde and applied to Hybond™-N membranes (Amersham) using a slot blot apparatus (BioRad). Prehybridization was performed at 42° C. for 1-3 h in 40-50% formamide in a solution containing 25 mM sodium phosphate, pH 7.4, 0.9 M sodium chloride, 50 mM sodium citrate, 0.1% each of gelatin, ficoll, and polyvinylpyrollidone, 0.5% SDS, and 100 μg/ml denatured salmon sperm DNA. Hybridization was carried out at the same temperature for 20h in the same solution with ³²P-labeled aP2 cDNA probe (2×10⁶ cpm/ml). After washing the membranes under appropriately stringent conditions, the hybridization signals were analyzed with a Phosphorlmager (Molecular Dynamics).

In Vivo Studies

Methods are described below.

Results

Binding Assay

Compound I is a potent PPARγ ligand. It displaces [³H]₂ thiazolidinedione binding to PPARγ with an IC50=23 nM.

PPAR-Gamma-GAL4 Transactivation Assay

Compound I was a partial agonist in this assay. It reached a maximal level of activity which was 25-35% of that achieved with full agonists such as rosiglitazone.

PPAR-CBP HTRF Assay

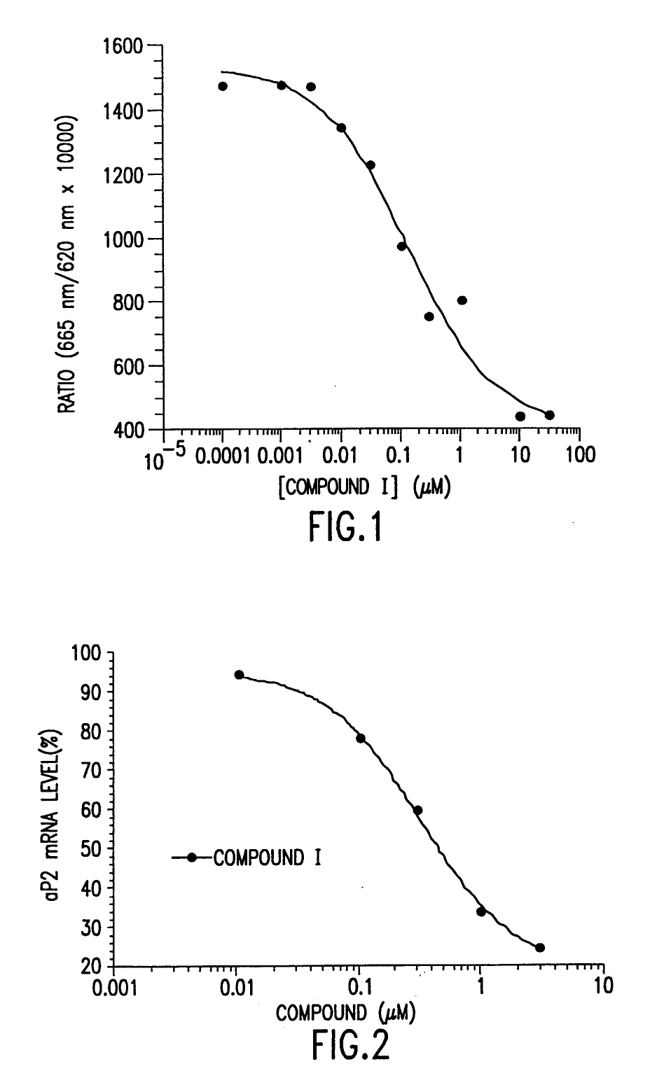

Compound I is a potent antagonist in the HTRF assay. When titrated in the presence of a known full agonist of PPARγ (3-chloro-4-(3-(3-phenyl-7-propylbenzofuran-6-yloxy)propylthio)phenylacetic acid at 60 nM), the agonist-induced PPARγ-CBP interaction is inhibited with an IC₅₀=133 nM (see FIG. 1). In addition, Compound I when tested alone displayed no significant (<5%) agonist activity.

Measurement of 3T3-L1 Preadipocyte Differentiation

Compound I served as a potent antagonist of PPARγ agonist-induced 3T3-L1 cell adipogenesis. Compound I blocked 100 nM rosiglitazone stimulated—aP2 expression with IC₅₀˜300 nM. (see FIG. 2)

In Vivo Studies—Methods and Results

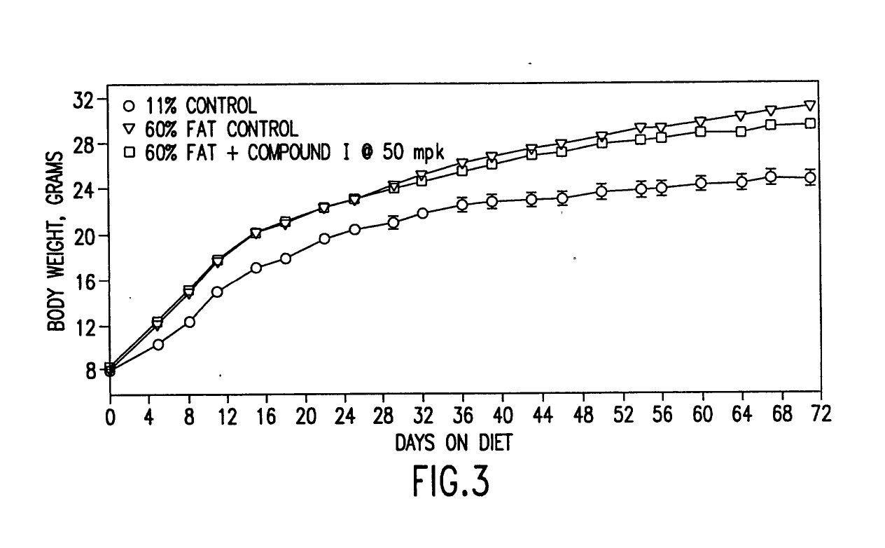

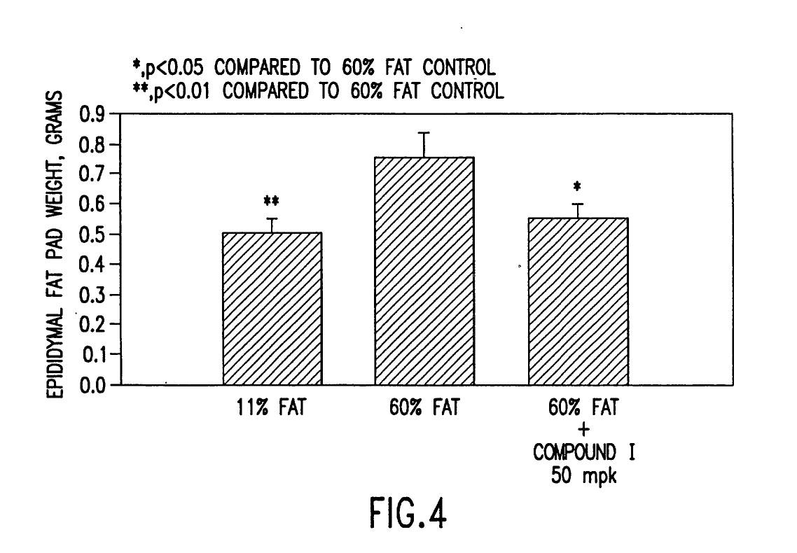

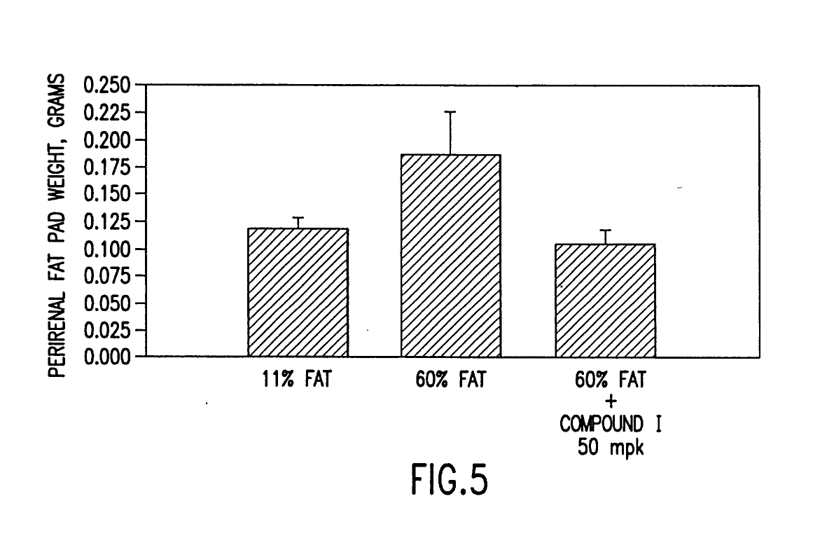

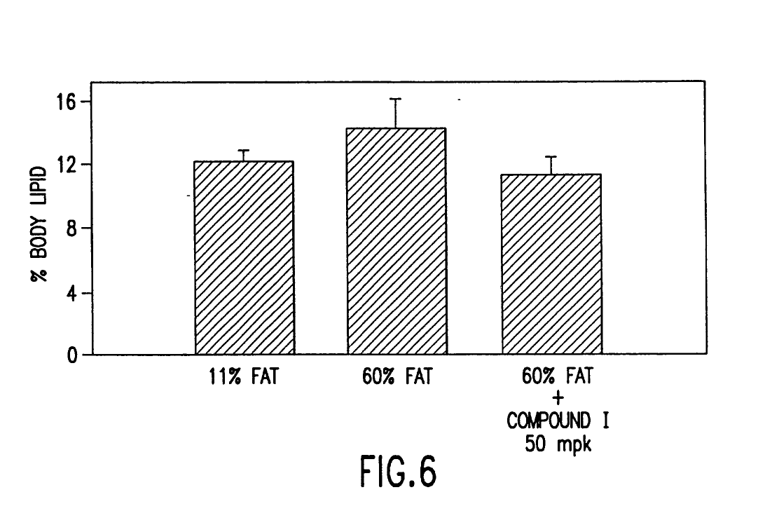

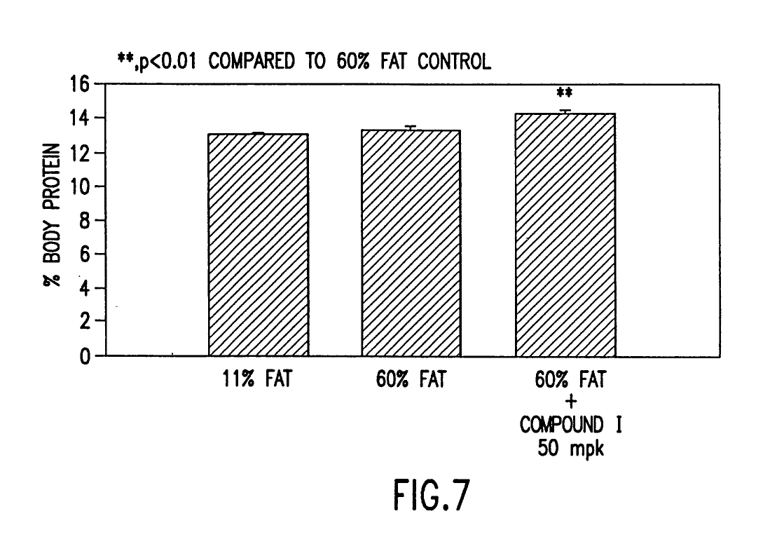

C57BL/6J mice were fed low fat (LF;11%), high fat (HF;58%) or HF+50 mg/kg/day Compound I diets for 72 days from the age of 18 days. At the end of that time the animals had gained 16g, 22 g and 20.5 g, respectively (FIG. 3). If the LF group is taken as basal or normal weight gain, these data describe a 25% decrease in excess of HF-induced weight in the HF+Compound I group relative to the HF group. When epididymal fat pad weights of the animals were examined, the HF group showed greater than 1.5-fold increase compared to the LF group. In contrast, the epididymal fat pad weight of the HF+Compound I mice was not significantly changed (see FIG. 4). Comparable effects of the PPARγ antagonist were noted when perirenal adipose tissue was examined (FIG. 5). Analysis of whole body triglyceride content and protein content were performed as described in Moller et al (Endocrinology 137:2397-2405, 1996). As shown in FIG. 6, there was a trend towards increased whole body triglyceride content in the HF vs. LF group whereas no increase in triglyceride content was evident in the HF+Compound I group. FIG. 7 shows that Compound I also increased total body protein in HF mice. Thus, treatment with the PPARγ antagonist caused relative decreases in body adiposity and increases in lean body mass in fat fed mice. The plasma levels of glucose, triglycerides and free fatty acid levels were all increased in the HF group relative to the LF group (see Table below). The HF+Compound I group demonstrated metabolic parameters similar to or even lower (TG) than the LF group (Table below).

TABLE 1
Effect of the PPARγ Antagonist (Comp'd I) on
Metabolic Parameters in Fat-fed Mice

| | | | HF Diet + |
|---|---|---|---|
| | | | 50 mg/kg/day |
| Parameter, units | Low fat diet | High fat Diet | Compound I |
| Plasma glucose | 137 +/− 4.9 | 64 +/− 7.5 | 145.6 +/− 4.1* |
| (mg/dl) | | | |
| Plasma tri- | 88.9 +/− 7.0 | 102.6 +/− 5.7 | 71.4 +/− 4.1** |
| glycerides, mg/dl | | | |
| Plasma free fatty | 0.52 +/− 0.052 | 0.728 +/− 0.065 | 0.536 +/− 0.02* |
| acids, mM | | | |
| Serum insulin, | 1.17 +/− 0.17 | 1.82 +/− 0.39 | 1.35 +/− 0.21 |
| ng/ml | | | |

Values are mean +/− SE of 10 mice.
*p <0.05 vs high fat control;
**p <0.001 vs high fat control

We claim:

1. A method of treating, preventing or controlling obesity in a mammalian patient in need thereof which comprises administering to said patient a therapeutically effective amount of a PPAR-gamma antagonist/partial agonist, wherein said PPAR-gamma antagonist/partial agonist inhibits at least 50% of the agonism of a full PPAR-gamma agonist and optionally also exhibits residual PPAR-gamma agonism.

2. A method of reducing body weight in an overweight mammalian patient in need of body weight reduction which comprises administering to said patient a therapeutically effective amount of a PPAR-gamma antagonist/partial agonist as defined in claim 1.

3. A method of treating, preventing or controlling hyperglycemia in a mammalian patient in need thereof which comprises administering to said patient a therapeutically effective amount of a PPAR-gamma antagonist/partial agonist as defined in claim 1.

4. A method of treating, preventing or controlling hyperlipidemia in a mammalian patient in need thereof which comprises administering to said patient a therapeutically effective amount of a PPAR-gamma antagonist/partial agonist as defined in claim 1.

5. A method of treating, preventing or controlling hypercholesteremia in a mammalian patient in need thereof which comprises administering to said patient a therapeutically effective amount of a PPAR-gamma antagonist/partial agonist as defined in claim 1.

6. A method of treating or controlling or preventing the onset of non-insulin dependent diabetes mellitus in a mammalian patient in need thereof which comprises administering to said patient a therapeutically effective amount of a PPAR-gamma antagonist/partial agonist as defined in claim 1.

7. A method of treating, preventing or controlling atherosclerosis in a mammalian patient in need thereof which comprises administering to said patient a therapeutically effective amount of a PPAR-gamma antagonist/partial agonist as defined in claim 1.

8. A method of treating, preventing or controlling hypertriglyceridemia in a mammalian patient in need thereof which comprises administering to said patient a therapeutically effective amount of a PPAR-gamma antagonist/partial agonist as defined in claim 1.

9. A method of treating, preventing or controlling insulin resistance in a mammalian patient in need thereof which comprises administering to said patient a therapeutically effective amount of a PPAR-gamma antagonist/partial agonist as defined in claim 1.

10. A method of treating, preventing or controlling hyperinsulinemia in a mammalian patient in need thereof which comprises administering to said patient a therapeutically effective amount of a PPAR-gamma antagonist/partial agonist as defined in claim 1.

11. A method of treating, preventing or controlling inflammatory bowel disease or inflammatory conditions in a mammalian patient in need thereof which comprises administering to said patient a therapeutically effective amount of a PPAR-gamma antagonist/partial agonist as defined in claim 1.

12. A method of treating, controlling or preventing obesity and also treating or controlling or preventing the onset of non-insulin dependent diabetes in a mammalian patient in need thereof which comprises administering to said patient a therapeutically effective amount of a PPAR-gamma antagonist/partial agonist as defined in claim 1.

13. A method of treating, controlling or preventing obesity and also treating, controlling or preventing with the same medication in a mammalian patient in need thereof one or more conditions selected from the group consisting of hyperglycemia, hyperlipidemia, hypercholesteremia, atherosclerosis, hypertriglyceridemia, inflammatory bowel disease, irritable bowel syndrome, inflammation, vascular restenosis, dyslipidemia, and polycystic ovarian syndrome, which comprises administering to said patient a therapeutically effective amount of a PPAR-gamma antagonist/partial agonist as defined in claim 1.

14. The method as recited in claim 1, which comprises administering to said patient a therapeutically effective amount of a PPAR-gamma antagonist/partial agonist, wherein said PPAR-gamma antagonist/partial agonist exhibits partial agonism in the range of about 5% to about 50% of the agonism of rosiglitazone or 3-chloro-4-(3-(3-phenyl-7-propylbenzofuran-6-yloxy)propylthio)phenylacetic acid as measured using the PPAR-CBP HTRF assay.

15. The method as recited in claim 1, which comprises administering to said patient a therapeutically effective amount of a PPAR-gamma antagonist/partial agonist, wherein said PPAR-gamma antagonist/partial agonist exhibits partial agonism in the range of about 5% to about 25% of the agonism of rosiglitazone or 3-chloro-4-(3-(3-phenyl-7-propylbenzofuran-6-yloxy)propylthio)phenylacetic acid as measured using the PPAR-CBP HTRF assay.

16. The method as recited in claim 1, which comprises administering to said patient a therapeutically effective amount of a PPAR-gamma antagonist/partial agonist, wherein said PPAR-gamma antagonist/partial agonist inhibits the agonism of rosiglitazone or 3-chloro-4-(3-(3-phenyl-7-propylbenzofuran-6-yloxy)propylthio)phenylacetic acid by at least 50% as measured using the PPAR-CBP HTRF assay.

17. The method as recited in claim 1, which comprises administering to said patient a therapeutically effective amount of a PPAR-gamma antagonist/partial agonist, wherein said PPAR-gamma antagonist/partial agonist inhibits the agonism of rosiglitazone or 3-chloro-4-(3-(3-phenyl-7-propylbenzofuran-6-yloxy)propylthio)phenylacetic acid by at least 75% as measured using the PPAR-CBP HTRF assay.

18. The method as recited in claim 1, which comprises administering to said patient a therapeutically effective amount of a PPAR-gamma antagonist/partial agonist, wherein said PPAR-gamma antagonist/partial agonist exhibits partial agonism in the range of about 5% to about 50% of the agonism of rosiglitazone or 3-chloro-4-(3-(3-phenyl-7-propylbenzofuran-6-yloxy)propylthio)phenylacetic acid as measured using the GAL4 chimeric receptor transcriptional assay.

19. The method as recited in claim 1, which comprises administering to said patient a therapeutically effective amount of a PPAR-gamma antagonist/partial agonist, wherein said PPAR-gamma antagonist/partial agonist exhibits partial agonism in the range of about 5% to about 25% of the agonism of rosiglitazone or 3-chloro-4-(3-(3-phenyl-7-propylbenzofuran-6-yloxy)propylthio)phenylacetic acid as measured using the GALA chimeric receptor transcriptional assay.

20. The method as recited in claim 1, which comprises administering to said patient a therapeutically effective amount of a PPAR-gamma antagonist/partial agonist, wherein said PPAR-gamma antagonist/partial agonist inhibits the agonism of rosiglitazone or 3-chloro-4-(3-(3-phenyl-7-propylbenzofuran-6-yloxy)propylthio)phenylacetic acid by at least 50% as measured using the GAL4 chimeric receptor transcriptional assay.

21. The method as recited in claim 1, which comprises administering to said patient a therapeutically effective amount of a PPAR-gamma antagonist/partial agonist, wherein said PPAR-gamma antagonist/partial agonist inhibits the agonism of rosiglitazone or 3-chloro-4-(3-(3-phenyl-7-propylbenzofuran-6-yloxy)propylthio)phenylacetic acid by at least 75% as measured using the GAL4 chimeric receptor transcriptional assay.

22. The method as recited in claim 1, which comprises administering to said patient a therapeutically effective amount of a PPAR-gamma antagonist/partial agonist, wherein said PPAR-gamma antagonist/partial agonist exhibits partial agonism in the range of about 5% to about 50% of the agonism of rosiglitazone as determined by measuring 3T3-L1 preadipocyte differentiation in the presence of the PPAR-gamma antagonist/partial agonist and comparing the agonism with that of rosiglitazone.

23. The method as recited in claim 1, which comprises administering to said patient a therapeutically effective amount of a PPAR-gamma antagonist/partial agonist, wherein said PPAR-gamma antagonist/partial agonist exhibits partial agonism in the range of about 5% to about 25% of the agonism of rosiglitazone as determined by measuring 3T3-L1 preadipocyte differentiation in the presence of the PPAR-gamma antagonist/partial agonist and comparing the agonism with that of rosiglitazone.

24. The method as recited in claim 1, which comprises administering to said patient a therapeutically effective amount of a PPAR-gamma antagonist/partial agonist, wherein said PPAR-gamma antagonist/partial agonist inhibits the agonism of rosiglitazone by at least 50% as determined by measuring 3T3-L1 preadipocyte differentiation in the presence of both the PPAR-gamma antagonist/partial agonist and rosiglitazone.

25. The method as recited in claim 1, which comprises administering to said patient a therapeutically effective amount of a PPAR-gamma antagonist/partial agonist, wherein said PPAR-gamma antagonist/partial agonist inhibits the agonism of rosiglitazone by at least 75% as determined by measuring 3T3-L1 preadipocyte differentiation in the presence of both the PPAR-gamma antagonist/partial agonist and rosiglitazone.

26. A pharmaceutical composition comprising a PPAR-gamma antagonist/partial agonist as defined in claim 1 and a pharmaceutically acceptable carrier.

27. A pharmaceutical composition comprising a PPAR-gamma antagonist/partial agonist and a pharmaceutically acceptable carrier, wherein said PPAR-gamma antagonist/partial agonist is defined in claim 14.

28. A pharmaceutical composition comprising a PPAR-gamma antagonist/partial agonist and a pharmaceutically acceptable carrier, wherein said PPAR-gamma antagonist/partial agonist is defined in claim 15.

29. A pharmaceutical composition comprising a PPAR-gamma antagonist/partial agonist and a pharmaceutically acceptable carrier, wherein said PPAR-gamma antagonist/partial agonist is defined in claim 16.

30. A pharmaceutical composition comprising a PPAR-gamma antagonist/partial agonist and a pharmaceutically acceptable carrier, wherein said PPAR-gamma antagonist/partial agonist is defined in claim 17.

31. A pharmaceutical composition comprising a PPAR-gamma antagonist/partial agonist and a pharmaceutically acceptable carrier, wherein said PPAR-gamma antagonist/partial agonist is defined in claim 18.

32. A pharmaceutical composition comprising a PPAR-gamma antagonist/partial agonist and a pharmaceutically acceptable carrier, wherein said PPAR-gamma antagonist/partial agonist is defined in claim 19.

33. A pharmaceutical composition comprising a PPAR-gamma antagonist/partial agonist and a pharmaceutically acceptable carrier, wherein said PPAR-gamma antagonist/partial agonist is defined in claim 20.

34. A pharmaceutical composition comprising a PPAR-gamma antagonist/partial agonist and a pharmaceutically acceptable carrier, wherein said PPAR-gamma antagonist/partial agonist is defined in claim 21.

35. A pharmaceutical composition comprising a PPAR-gamma antagonist/partial agonist and a pharmaceutically acceptable carrier, wherein said PPAR-gamma antagonist/partial agonist is defined in claim 22.

36. A pharmaceutical composition comprising a PPAR-gamma antagonist/partial agonist and a pharmaceutically acceptable carrier, wherein said PPAR-gamma antagonist/partial agonist is defined in claim 23.

37. A pharmaceutical composition comprising a PPAR-gamma antagonist/partial agonist and a pharmaceutically acceptable carrier, wherein said PPAR-gamma antagonist/partial agonist is defined in claim 24.

38. A pharmaceutical composition comprising a PPAR-gamma antagonist/partial agonist and a pharmaceutically acceptable carrier, wherein said PPAR-gamma antagonist/partial agonist is defined in claim 25.

39. A method of evaluating a PPAR-gamma ligand as an anti-obesity and/or anti-diabetic agent in vivo comprising the steps of (1) administering a PPAR-gamma ligand to one or more C57BL/6J mice for a period of at least 14 days, and (2) measuring the effect of the PPAR-gamma ligand on one or more parameters that characterize obesity and/or diabetes.

40. The method as recited in claim 39, wherein at least one parameter is selected from the group consisting of body weight, epididymal fat pad weight, perirenal fat pad weight, whole body triglyceride content, whole body protein content, body adiposity, lean body mass, plasma glucose level, plasma triglyceride level, plasma free fatty acid level, and serum insulin level.

41. A method for selecting a compound for in vivo testing as an anti-obesity agent, comprising the steps of (1) providing a candidate compound, and (2) measuring the PPAR-gamma antagonism of the candidate compound in the presence of a full PPAR-gamma agonist.

42. The method as recited in claim 41, wherein the full PPAR-gamma agonist is rosiglitazone or 3-chloro-4-(3-(3-phenyl-7-propylbenzofuran-6-yloxy)propylthio)phenylacetic acid.

43. The method as recited in claim 41, wherein the compound is part of a collection of at least 10 candidate compounds, and the compound having the highest % inhibition of the full PPAR-gamma agonist is selected for testing as an anti-obesity agent.

44. The method as recited in claim 41, wherein the PPAR-gamma antagonism is measured using an assay method selected from the group consisting of the PPAR-CBP HTRF assay, the GAL-4 chimeric receptor transcriptional assay, and 3T3-L1 preadipocyte differentiation.

45. A method for selecting a compound for in vivo testing as an anti-obesity agent which also has anti-diabetic activity, comprising the steps of (1) providing a candidate compound, and (2) measuring the PPAR-gamma partial agonism of the candidate compound compared with a full PPAR-gamma agonist.

46. The method as recited in claim 45, wherein the full PPAR-gamma agonist is rosiglitazone or 3-chloro-4-(3-(3-phenyl-7-propylbenzofuran-6-yloxy)propylthio)phenylacetic acid.

47. The method as recited in claim 45, wherein the compound is part of a collection of at least 10 candidate compounds.

48. The method as recited in claim 45, wherein the compounds selected for in vivo testing have a PPAR-gamma partial agonism in the range of about 5% to about 25% of the agonism of rosiglitazone.

49. The method as recited in claim 45, wherein the PPAR-gamma partial agonism is measured using an assay method selected from the group consisting of the PPAR-CBP HTRF assay, the GAL-4 chimeric receptor transcriptional assay, and 3T3-L1 preadipocyte differentiation.

50. A method of measuring the partial agonism of a PPAR-gamma antagonist, comprising the step of measuring the inhibition of a full agonist by the PPAR-gamma antagonist using the PPAR-CBP HTRF assay, and measuring the residual agonism of the PPAR-gamma ligand during the same assay, wherein the residual agonism is the partial agonism of the PPAR-gamma antagonist.

51. A method of treating, preventing or controlling obesity in a mammalian patient in need thereof which comprises administering to said patient a therapeutically effective amount of a compound defined by Formula II, below, or a pharmaceutically acceptable salt thereof: 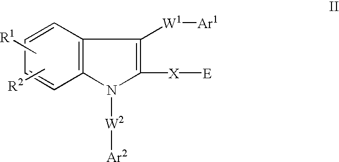
wherein, R¹ and R² are independently selected from H, halogen, C₁₋₁₀ alkyl, C₂₋₁₀ alkenyl and C₁₋₁₀ alkoxy, where the alkyl, alkenyl, and alkoxy groups are optionally substituted with 1-3 groups independently selected from R^{a}, except that the number of optional F groups where R^{a} is F is in the range of 1-21;
R^{a} is selected from OH, halogen, C₁₋₃ alkyl, C₁₋₃ alkyl having 1-7 halogen atom substituents, C₁₋₃ alkoxy, C₁₋₃ alkoxy having 1-7 halogen atom substituents, phenyl, and phenyl substituted with 1-3 groups independently selected from halogen, OCH₃, OCF₃, CH₃, and CF_{3;}
Ar¹ and Ar² are each independently selected from the group consisting of aryl and heteroaryl, wherein Ar¹ and Ar² are optionally substituted with 1-3 substituents independently selected from R^{a} and are optionally substituted with one COOH group;
X, W¹ and W² are each independently selected from the group consisting of a single bond, Y or Y(CH₂)ₙY¹, where Y and Y¹ are each independently selected from the group consisting of a single bond, O, S, SO, SO₂, and NR;
n is 1-3;
R is selected from H, C₁₋₃ alkyl and C₂₋₃ alkenyl, where the alkyl and alkenyl groups are optionally substituted with 1-7 halogen atoms and/or 1-3 groups selected from OH, C₁₋₃ alkoxy, and C₁₋₃ alkoxy substituted with 1-7 halogen atoms;
and E is CO₂H, C(O)NR₂, or a tetrazol-5-yl, where each R is independently defined above.

52. A method of reducing body weight in an overweight mammalian patient in need of body weight reduction which comprises administering to said patient a therapeutically effective amount of a compound defined by Formula II, as defined in claim 51, or a pharmaceutically effective salt thereof.

53. A method of treating, preventing or controlling hyperglycemia in a mammalian patient in need thereof which comprises administering to said patient a therapeutically effective amount of a compound defined by Formula II, as defined in claim 51, or a pharmaceutically effective salt thereof.

54. A method of treating, preventing or controlling hyperlipidemia in a mammalian patient in need thereof which comprises administering to said patient a therapeutically effective amount of a compound defined by Formula II, as defined in claim 51, or a pharmaceutically effective salt thereof.

55. A method of treating, preventing or controlling hypercholesteremia in a mammalian patient in need thereof which comprises administering to said patient a therapeutically effective amount of a compound defined by Formula II, as defined in claim 51, or a pharmaceutically effective salt thereof.

56. A method of treating, controlling or preventing the onset of non-insulin dependent diabetes mellitus in a mammalian patient in need thereof which comprises administering to said patient a therapeutically effective amount of a compound defined by Formula II, as defined in claim 51, or a pharmaceutically effective salt thereof.

57. A method of treating, preventing or controlling atherosclerosis in a mammalian patient in need thereof which comprises administering to said patient a therapeutically effective amount of a compound defined by Formula II, as defined in claim 51, or a pharmaceutically effective salt thereof.

58. A method of treating, preventing or controlling hypertriglyceridemia in a mammalian patient in need thereof which comprises administering to said patient a therapeutically effective amount of a compound defined by Formula II, as defined in claim 51, or a pharmaceutically effective salt thereof.

59. A method of treating, preventing or controlling insulin resistance in a mammalian patient in need thereof which comprises administering to said patient a therapeutically effective amount of a compound defined by Formula II, as defined in claim 51, or a pharmaceutically effective salt thereof.

60. A method of treating, preventing or controlling hyperinsulinemia in a mammalian patient in need thereof which comprises administering to said patient a therapeutically effective amount of a compound defined by Formula II, as defined in claim 51, or a pharmaceutically effective salt thereof.

61. A method of treating, preventing or controlling inflammatory bowel disease or an inflammatory condition in a mammalian patient in need thereof which comprises administering to said patient a therapeutically effective amount of a compound defined by Formula II, as defined in claim 51, or a pharmaceutically effective salt thereof.

62. A method of treating, controlling or preventing obesity and also treating or controlling or preventing the onset of non-insulin dependent diabetes in a mammalian patient in need thereof which comprises administering to said patient a therapeutically effective amount of a compound defined by Formula II, as defined in claim 51, or a pharmaceutically effective salt thereof.

63. A method of treating, controlling or preventing obesity and also treating, controlling or preventing with the same medication in a mammalian patient in need thereof one or more conditions selected from the group consisting of hyperglycemia, hyperlipidemia, hypercholesteremia, atherosclerosis, hypertriglyceridemia, inflammatory bowel disease, irritable bowel syndrome, inflammation, vascular restenosis, dyslipidemia, polycystic ovarian syndrome, and cancer, which comprises administering to said patient a therapeutically effective amount of a compound defined by Formula II, as defined in claim 51, or a pharmaceutically effective salt thereof.

64. A method as recited in claim 51, wherein the substituents in Formula II are defined as follows:
R¹ is H, C₁₋₃ alkyl, C₁₋₃ alkoxy, or halogen;
R² is H;
W¹ and W² are each a single bond, O, S, SO, SO₂, NH, or CH₂;
X is a bond or CH_{2;}
Ar¹ and Ar² are each aryl, optionally substituted with 1-2 substituents independently selected from halogen, methoxy, and C13 alkyl; and
E is CO₂H.

65. A method as recited in claim 51, wherein heteroaryl is selected from the group consisting of thiophene, pyrrole, furan, or pyridine, and aryl is phenyl.

66. A method of treating, preventing or controlling obesity in a mammalian patient in need thereof which comprises administering to said patient a therapeutically effective amount of a compound defined by Formula I, or a pharmaceutically acceptable salt thereof:

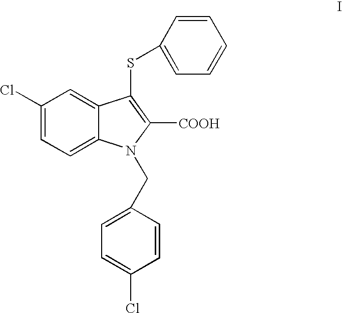

67. A method of reducing body weight in a mammalian patient in need of body weight reduction which comprises administering to said patient a therapeutically effective amount of a compound having Formula I as recited in claim 66, or a pharmaceutically acceptable salt thereof.

68. A method of treating, preventing or controlling one or more conditions selscted from the group consisting of hyperglycemia, hyperlipidemia, hypercholesteremia, non-insulin dependent diabetes mellitus, atherosclerosis, hypertriglyceridemia, insulin resistance, hyperinsulinemia, inflammatory bowel disease and other inflammatory conditions, vascular restenosis, dyslipidemia and polycystic ovarian syndrome, which comprises administering to a patient in need of such treatment a therapeutically effective amount of the compound of claim 66 having Formula I, or a pharmaceutically effective salt thereof.

69. A pharmaceutical composition comprising a compound of Formula II, as defined in claim 50, or a pharmaceutically acceptable salt thereof, and a pharmaceutically acceptable carrier.

70. A pharmaceutical composition comprising Compound II, as defined in claim 64, or a pharmaceutically acceptable salt thereof, and a pharmaceutically acceptable carrier.

71. A pharmaceutical composition comprising a compound defined in claim 66, or a pharmaceutically acceptable salt thereof, in a pharmaceutically acceptable carrier.

72. A method of treating, controlling or preventing obesity, of reducing body weight, of treating, controlling or preventing the onset of non-insulin dependent diabetes mellitus, or of treating, controlling or preventing one or more other conditions selected from the group consisting of hyperglycemia, hyperlipidemia, hypercholesteremia, atherosclerosis, hypertriglyceridemia, insulin resistance, hyperinsulinemia, inflammatory bowel disease, irritable bowel syndrome, inflammation, vascular restenosis, dyslipidemia, and polycystic ovarian syndrome in a mammalian patient in need of treatment comprising the administration of a therapeutically effective amount of a prodrug of the compound having formula II, as defined in claim 51.

73. A method of treating, controlling or preventing obesity, of reducing body weight, of treating, controlling or preventing the onset of non-insulin dependent diabetes mellitus, or of treating, controlling or preventing one or more other conditions selected from the group consisting of hyperglycemia, hyperlipidemia, hypercholesteremia, atherosclerosis, hypertriglyceridemia, insulin resistance, hyperinsulinemia, inflammatory bowel disease, irritable bowel syndrome, inflammation, vascular restenosis, dyslipidemia, and polycystic ovarian syndrome in a mammalian patient in need of treatment comprising the administration of a therapeutically effective amount of a prodrug of the compound having formula II, as defined in claim 64.

74. A method of treating, controlling or preventing obesity, of reducing body weight, of treating, controlling or preventing the onset of non-insulin dependent diabetes mellitus, or of treating, controlling or preventing one or more other conditions selected from the group consisting of hyperglycemia, hyperlipidemia, hypercholesteremia, atherosclerosis, hypertriglyceridemia, insulin resistance, hyperinsulinemia, inflammatory bowel disease, irritable bowel syndrome, inflammation, vascular restenosis, dyslipidemia, and polycystic ovarian syndrome in a mammalian patient in need of treatment comprising the administration of a therapeutically effective amount of a prodrug of the compound having formula I, as defined in claim 66.
